(19) United States
(12) Patent Application Publication
(10) Pub. No.: US 2024/0300901 A1
(43) Pub. Date: Sep. 12, 2024

(54) METHODS FOR THE PREPARATION OF 5-BROMO-2-(3-CHLORO-PYRIDIN-2-YL)-2H-PYRAZOLE-3-CARBOXYLIC ACID

(71) Applicants: FMC Corporation, Philadelphia, PA (US); FMC AGRO SINGAPORE PTE. LTD., Singapore (SG)

(72) Inventors: Yanchun CAO, Shanghai (CN); Zhijian XU, Shanghai (CN); Xin LIU, Shanghai (CN)

(21) Appl. No.: 18/274,354

(22) PCT Filed: Jan. 26, 2022
(86) PCT No.: PCT/US2022/013858

§ 371 Date: Jul. 26, 2023

Related U.S. Application Data
(60) Provisional application No. 63/143,156, filed on Jan. 29, 2021.

(51) Int. Cl. C07D 231/16; C07D 401/04

(52) U.S. Cl. CPC C07D 231/16; C07D 401/04

(57) ABSTRACT

Described herein are novel methods of synthesizing 5-Bromo-2-(3-chloro-pyridin-2-yl)-2H-pyrazole-3-carboxylic acid from pyrazole or pyrazole derivatives.

### CROSS-REFERENCE TO RELATED APPLICATIONS

This application claims the benefit of U.S. Provisional Application No. 63/143,156 filed Jan. 29, 2021.

### FIELD OF INVENTION

This disclosure is directed to novel methods of synthesizing 5-Bromo-2-(3-chloro-pyridin-2-yl)-2H-pyrazole-3-carboxylic acid. Compounds prepared by the methods disclosed herein are useful for preparation of certain anthranilamide compounds that are of interest as insecticides, such as, for example, the insecticides chlorantraniliprole and cyantraniliprole.

### BACKGROUND

Conventional processes for the production of 5-Bromo-2-(3-chloro-pyridin-2-yl)-2H-pyrazole-3-carboxylic acid are subject to several industrial concerns, such as processability, environmental hazards, high cost, reagent reactivity, and necessary specialized equipment.

The present disclosure provides novel methods useful for preparing 5-Bromo-2-(3-chloro-pyridin-2-yl)-2H-pyrazole-3-carboxylic acid and derivatives thereof. The benefits of the methods of the present disclosure compared to previous methods are numerous and include improved overall yield, reduced cost, eliminated need for mixed solvent separations, reduced waste, simplified operation complexity, and reduced process hazards.

### BRIEF DESCRIPTION

In one aspect, provided herein is a method of preparing a compound of Formula V, wherein

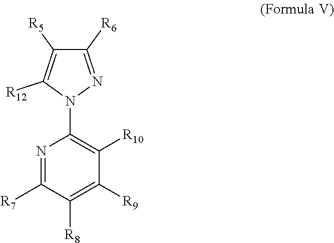

each of R₅-R₁₀ is independently selected from hydrogen and halogen; and
R₁₂ is an organic acid, the method comprising
   I) forming a mixture comprising
      A) a compound of Formula III, wherein

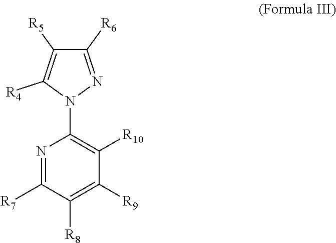

R₄ is a halogen; and
each of R₅-R₁₀ is independently selected from hydrogen and halogen;
B) a solvent;
C) a carbonyl-containing compound;
D) a compound comprising a metal; and
E) optionally an additive; and
II) reacting the mixture.

In one aspect, provided herein is a method of preparing a compound of Formula II, wherein

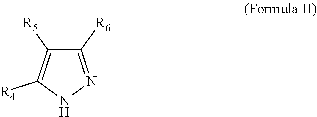

each of R₄, R₅, and R₆ is independently selected from hydrogen and halogen; and
wherein at least one of R₄, R₅, and R₆ is hydrogen, the method comprising
   I) forming a mixture comprising
      A) a compound of Formula I, wherein

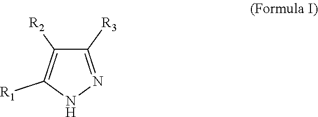

each of R₁, R₂, and R₃ is independently a halogen; and
wherein the compound of Formula I is prepared according to a method comprising
i) forming a mixture comprising
a) pyrazole or a pyrazole derivative;
b) a halogenation reagent;
c) a reaction solvent comprising water and optionally an organic solvent; and
d) optionally an inorganic base; and
ii) reacting the mixture;
B) optionally a dehalogenation reagent;
C) a reducing agent; and
D) a solvent; and
II) reacting the mixture.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

When a group contains a substituent which can be hydrogen, for example R⁴, then, when this substituent is taken as hydrogen, it is recognized that this is equivalent to said group being unsubstituted.

The term “organic base” includes, without limitation, amine compounds (e.g., primary, secondary and tertiary amines), heterocycles including nitrogen-containing heterocycles, and ammonium hydroxide.

The term “inorganic base” includes, without limitation, inorganic compounds with the ability to react with, or neutralize, acids to form salts, such as, for example, metal salts of hydroxide, carbonate, bicarbonate and phosphate.

The term “halogenation reagent” includes, without limitation, halogens and inorganic compounds, such as, for example, bromine, NBS, and 1,3-dibromo-5,5-dimethylhylhydantoin.

The term “phase transfer catalyst” includes compounds that facilitate the migration of a reactant from one phase into another phase where a reaction occurs. Phase transfer catalysis refers to the acceleration of the reaction upon the addition of the phase transfer catalyst.

The term “ether” includes, without limitation, a functional group comprising an ether bond (C—O—C).

The term “carboxylic acid” includes, without limitation, a functional group comprising a carboxylic acid bond (C(═O)—OH).

The term “organic acid” includes, without limitation, a functional group that confers acidity and consists of atoms selected from carbon, nitrogen, oxygen, and hydrogen.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula V, wherein

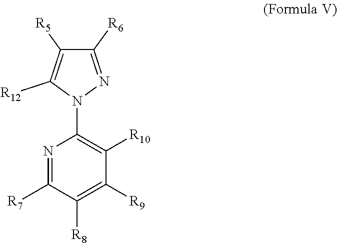

each of R₅-R₁₀ is independently selected from hydrogen and halogen; and
R₁₂ is an organic acid, the method comprising
   I) forming a mixture comprising
      A) a compound of Formula III, wherein

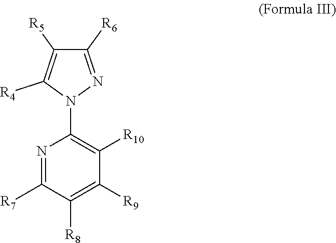

R₄ is a halogen; and
each of R₅-R₁₀ is independently selected from hydrogen and halogen;
B) a solvent;
C) a carbonyl-containing compound;
D) a compound comprising a metal; and
E) optionally an additive; and
II) reacting the mixture.

Embodiment 2. The method of embodiment 1, wherein the compound comprising a metal is selected from a Grignard reagent and a lithium-containing compound.

Embodiment 3. The method of embodiment 2, wherein the Grignard reagent is selected from MeMgCl, iPrMgCl, iPrMgBr, EtMgCl, and combinations thereof.

Embodiment 4. The method of embodiment 3, wherein the Grignard reagent is iPrMgCl.

Embodiment 5. The method of embodiment 2, wherein the lithium-containing compound is nBuLi.

Embodiment 6. The method of embodiment 1, wherein the solvent is selected from THF, toluene, 1,4-dioxane, Me-THF, and combinations thereof.

Embodiment 7. The method of embodiment 6, wherein the solvent is THF.

Embodiment 8. The method of embodiment 1, wherein the carbonyl-containing compound is selected from dimethyl carbonate, N,N-dimethyacetamide, carbon dioxide, and combinations thereof.

Embodiment 9. The method of embodiment 8, wherein the carbonyl-containing compound is carbon dioxide.

Embodiment 10. The method of embodiment 1, wherein the method step II) of reacting the mixture occurs at a reaction temperature in the range of about 0° C. to about 60° C.

Embodiment 11. The method of embodiment 10, wherein the method step II) of reacting the mixture occurs at a reaction temperature in the range of about 0° C. to about 30° C.

Embodiment 12. The method of embodiment 1, wherein R₅ and R₆ of Formula III are each independently hydrogen.

Embodiment 13. The method of embodiment 1, wherein the compound of Formula III is prepared according to a method comprising
I) forming a mixture comprising
   A) a compound of Formula II, wherein

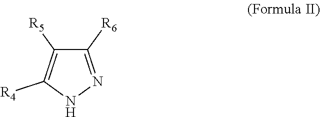

each of R₄, R₅, and R₆ is independently selected from hydrogen and halogen;
wherein at least one of R₄, R₅, and R₆ is hydrogen; and
wherein the compound of Formula II is prepared according to a method comprising
   i) forming a mixture comprising
   a) a compound of Formula I, wherein

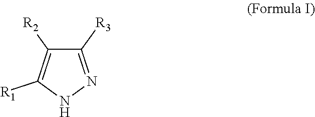

each of R₁, R₂, and R₃ is independently a halogen;
b) optionally a dehalogenation reagent;
c) a reducing agent; and
d) a solvent; and
ii) reacting the mixture;
B) a compound of Formula IV, wherein

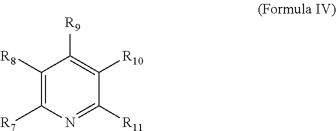

each of R₇-R₁₁ is independently selected from hydrogen and halogen;
C) a solvent;
D) an inorganic base; and
E) optionally an additive; and
II) reacting the mixture.

Embodiment 14. The method of embodiment 13, wherein the inorganic base is selected from powder sodium hydroxide, powder potassium hydroxide, potassium carbonate, potassium phosphate, powder sodium methoxide, powder potassium t-butoxide, and combinations thereof.

Embodiment 15. The method of embodiment 13, wherein the solvent C) is selected from the solvent is selected from heavy aromatic solvents, heavy aromatic solvent S150, heavy aromatic solvent S200, acetonitrile (MeCN), toluene, N,N-dimethylformamide (DMF), N,N-dimethylacetamide (DMAc), N-methyl-2-pyrrolidone (NMP), diglyme, triglyme, sulfolane and combinations thereof.

Embodiment 16. The method of embodiment 13, wherein the additive is selected from potassium iodide, a phase transfer catalyst, and combinations thereof.

Embodiment 17. The method of embodiment 16, wherein the phase transfer catalyst is selected from butyl ammonium chloride, tetra butyl ammonium bromide, aliquat-336, 18-crown-6, and combinations thereof.

Embodiment 18. The method of embodiment 13, wherein the method step II) of reacting the mixture occurs at a reaction temperature in the range of about 140° C. to about 200° C.

Embodiment 19. The method of embodiment 13, wherein the solvent d) is selected from acetic acid, water, toluene, N,N-dimethylformamide, N,N-dimethylacetamide, and combinations thereof.

Embodiment 20. The method of embodiment 13, wherein the reducing agent is selected from sodium sulfite, sodium bisulfite, sodium hyposulfite, sodium thiosulfate, sodium hydrosulfide, sodium sulfate, and combinations thereof.

Embodiment 21. The method of embodiment 13, wherein the dehalogenation reagent is selected from sodium iodide, iodine, potassium iodide, tetra-n-butyl ammonium iodide, and combinations thereof.

Embodiment 22. The method of embodiment 13, wherein the method step ii) of reacting the mixture occurs at a reaction temperature in the range of about 100° C. to about 180° C.

Embodiment 23. The method of embodiment 13, wherein the compound of Formula I is prepared according to a method comprising
I) forming a mixture comprising
   A) pyrazole or a pyrazole derivative;
   B) a halogenation reagent;
   C) a reaction solvent comprising water and optionally an organic solvent; and
   D) optionally an inorganic base; and
II) reacting the mixture.

Embodiment 24. The method of embodiment 23, wherein the halogenation reagent comprises
A) a reagent selected from hydrogen bromide, bromine, N-bromosuccinimide, 1,3-dibromo-5,5-dimethylhylhydantoin, sodium bromide, potassium bromide, and combinations thereof, and
B) optionally hydrogen peroxide.

Embodiment 25. The method of embodiment 23, wherein the inorganic base is selected from powder sodium hydroxide, sodium hydroxide solution, powder sodium acetate, and combinations thereof.

Embodiment 26. The method of embodiment 23, wherein the method step II) of reacting the mixture occurs at a reaction temperature in the range of about 0° C. to about 70° C.

Embodiment 27. A method of preparing a compound of Formula II, wherein

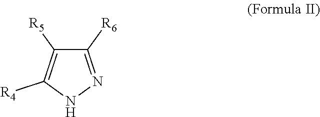

each of R₄, R₅, and R₆ is independently selected from hydrogen and halogen; and
wherein at least one of R₄, R₅, and R₆ is hydrogen, the method comprising
   I) forming a mixture comprising
      A) a compound of Formula I, wherein

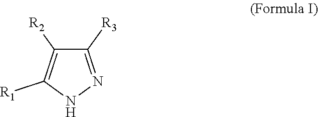

each of R₁, R₂, and R₃ is independently a halogen; and
wherein the compound of Formula I is prepared according to a method comprising
i) forming a mixture comprising
a) pyrazole or a pyrazole derivative;
b) a halogenation reagent;
c) a reaction solvent comprising water and an organic solvent; and
d) optionally an inorganic base; and
ii) reacting the mixture;
B) optionally a dehalogenation reagent;
C) a reducing agent; and
D) a solvent; and
II) reacting the mixture.

Embodiment 28. The method of embodiment 27, wherein the solvent D) is selected from acetic acid, water, toluene, N,N-dimethylformamide, N,N-dimethylacetamide, and combinations thereof.

Embodiment 29. The method of embodiment 27, wherein the reducing agent is selected from sodium sulfite, sodium bisulfite, sodium hyposulfite, sodium thiosulfate, sodium hydrosulfide, sodium sulfate, and combinations thereof.

Embodiment 30. The method of embodiment 27, wherein the dehalogenation reagent is selected from sodium iodide, iodine, potassium iodide, tetra-n-butyl ammonium iodide, and combinations thereof.

Embodiment 31. The method of embodiment 27, wherein the method step II) of reacting the mixture occurs at a reaction temperature in the range of about 100° C. to about 180° C.

Embodiment 32. The method of embodiment 27, wherein the halogenation reagent comprises
A) a reagent selected from hydrogen bromide, bromine, N-bromosuccinimide, 1,3-dibromo-5,5-dimethylhylhydantoin, sodium bromide, potassium bromide, and combinations thereof, and
B) optionally hydrogen peroxide.

Embodiment 33. The method of embodiment 27, wherein the inorganic base is selected from powder sodium hydroxide, sodium hydroxide solution, powder sodium acetate, and combinations thereof.

Embodiment 34. The method of embodiment 27, wherein the method step ii) of reacting the mixture occurs at a reaction temperature in the range of about 0° C. to about 70° C.

Embodiment 35. The method of embodiment 27, wherein the reaction solvent comprises an organic solvent selected from MBTE, ethanol, DCM, chloroform, and combinations thereof.

In one aspect, a compound of Formula V is prepared according to a method represented by Scheme 1. The R groups are as defined anywhere in this disclosure.

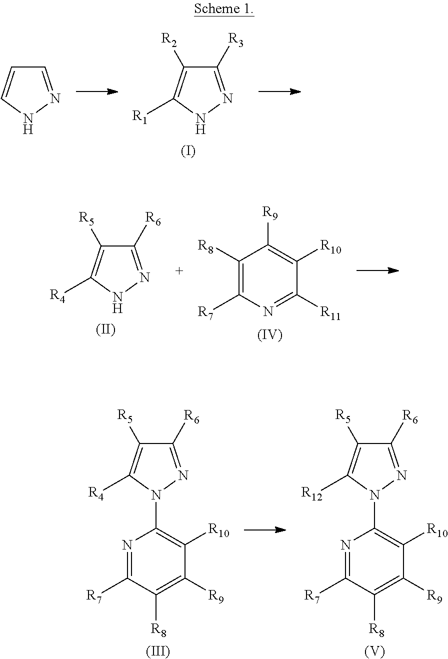

In one aspect, 5-Bromo-2-(3-chloro-pyridin-2-yl)-2H-pyrazole-3-carboxylic acid is prepared according to a method represented by Scheme 2.

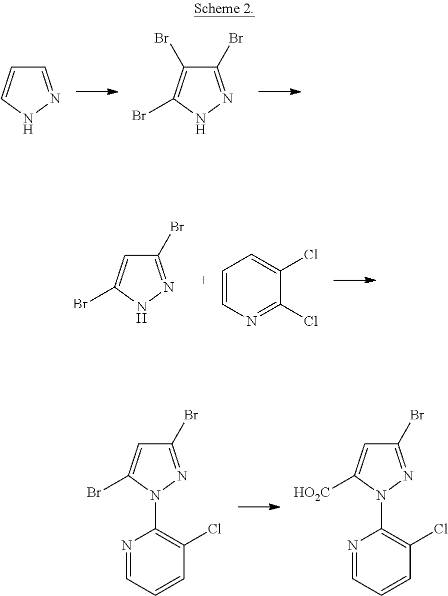 [SMILES: c1cn[nH]c1 ; Brc1n[nH]c(Br)c1Br ; Brc1cc(Br)[nH]n1 ; Clc1cccnc1Cl ; Clc1cccnc1-n1nc(Br)cc1Br ; O=C(O)c1cc(Br)nn1-c1ncccc1Cl]

In one aspect, a compound of Formula I is prepared according to a method represented by Scheme 3. The R groups are as defined anywhere in this disclosure.

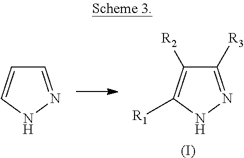

This aspect includes reacting pyrazole with a halogenation reagent in a reaction solvent including water and optionally an organic solvent, and optionally in the presence of an inorganic base. In one embodiment, the halogenation reagent is selected from hydrogen peroxide/HBr, Bromine (Br₂), N-bromosuccinimide, 1,3-dibromo-5,5-dimethylhylhydantoin, hydrogen peroxide/NaBr, hydrogen peroxide/KBr, hydrogen peroxide/Br₂, and combinations thereof. In another embodiment, the halogenation reagent is Br₂. In one embodiment, inorganic base is selected from powder sodium hydroxide, sodium hydroxide solution, powder sodium acetate, and combinations thereof. In another embodiment the inorganic base is power sodium hydroxide. In one embodiment, the reaction temperature is in the range from about 0° C. to about 70° C. In another embodiment, the reaction temperature is in the range from about 0° C. to about 30° C. In one embodiment, the organic solvent is selected from methyl tert-butyl ether (MTBE), ethanol, dichloromethane (DCM), chloroform, and combinations thereof. In another embodiment, the organic solvent is MTBE.

In one aspect, a compound of Formula II is prepared according to a method represented by Scheme 4. The R groups are as defined anywhere in this disclosure.

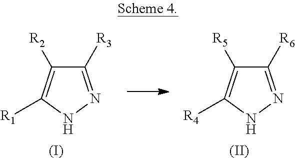

This aspect includes reacting a compound of Formula I with a dehalogenation reagent in a solvent in the presence of a reducing agent. In one embodiment, the solvent is selected from acetic acid, water, toluene, N,N-dimethylformamide (DMF), N,N-dimethylacetamide (DMAc), diglyme, sulfolane, and combinations thereof. In another embodiment, the solvent is N,N-dimethylacetamide (DMAc). In one embodiment, the dehalogenation reagent is selected from sodium iodide, iodine, potassium iodide, tetra-n-butyl ammonium iodide (TBAI), and combinations thereof. In another embodiment, the dehalogenation reagent is potassium iodide. In one embodiment, the reducing agent is selected from sodium sulfite, sodium bisulfite, sodium hyposulfite, sodium thiosulfate, sodium hydrosulfide, sodium sulphate, and combinations thereof. In another embodiment, the reducing agent is sodium sulfite. In one embodiment, the reaction temperature is in the range from about 100° C. to about 180° C. In another embodiment, the reaction temperature is in the range from about 130° C. to about 150° C.

In one aspect, a compound of Formula III is prepared according to a method represented by Scheme 5. The R groups are as defined anywhere in this disclosure.

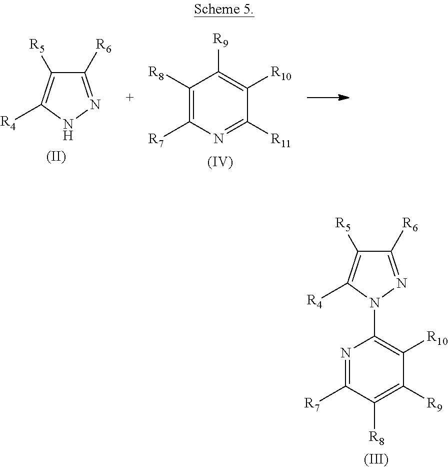

This aspect includes the step of mixing a compound of Formula II with a compound of Formula IV in a solvent in the presence of an inorganic base and optionally an additive. In one embodiment, the inorganic base is selected from powder sodium hydroxide, powder potassium hydroxide, potassium carbonate, sodium carbonate, potassium phosphate powder sodium methoxide, powder potassium t-butoxide, and combinations thereof. In another embodiment, the inorganic base is sodium carbonate. In one embodiment, the solvent is selected from heavy aromatic solvents, heavy aromatic solvent S150, heavy aromatic solvent S200, acetonitrile (MeCN), toluene, N,N-dimethylformamide (DMF), N,N-dimethylacetamide (DMAc), N-methyl-2-pyrrolidone (NMP), diglyme, triglyme, sulfolane and combinations thereof. In another embodiment, the solvent is sulfolane. In one embodiment, the additive is a phase catalyst selected from butyl ammonium chloride (TBAC), tetra butyl ammonium bromide (TBAB), aliquat-336, 18-crown-6, and combinations thereof. In another embodiment, the phase catalyst is 18-crown-6. In another embodiment, the additive is potassium iodide. In one embodiment, the reaction temperature ranging is in the range from about 140° C. to about 200° C. In another embodiment, the temperature is in the range from about 170° C. to about 180° C.

In one aspect, a compound of Formula V is prepared according to a method represented by Scheme 6. The R groups are as defined anywhere in this disclosure.

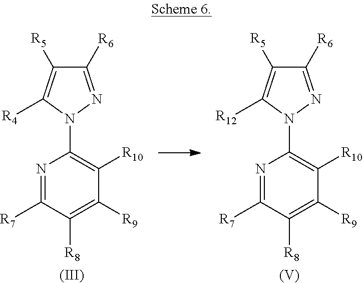

This aspect includes mixing a compound of Formula III with a carbonyl-containing compound in a solvent in the presence of a base reagent and optionally an additive. In one embodiment, the carbonyl-containing compound is selected from dimethylcarbonate, N,N-dimethylacetamide, carbon dioxide (CO₂), and combinations thereof. In another embodiment, the carbonyl-containing compound is CO₂. In one embodiment, the base reagent is selected from MeMgCl, iPrMgCl, iPrMgBr, EtMgCl, LDA, nBuLi, iPr₂NMgCl, iPr₂NMgBr, Et2NMgCl, TMPMgCl, iPr₂NMgCl·LiCl, iPr₂NMgBr·LiCl, and combinations thereof. In another embodiment, the base reagent is iPrMgCl. In one embodiment, the solvent is selected from tetrahydrofuran (THF), toluene, 1,4-dioxane, 2-methyltetrahydrofuran (Me-THF), and combinations thereof. In another embodiment, the solvent is THF. In one embodiment, the reaction temperature is in the range from about 0° C. to about 60° C. In another embodiment, the temperature is in the range from about 0° C. to about 30° C.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Hydrogen Peroxide/HBr as a Halogenation Reagent

34 grams of pyrazole and 505.8 g of 48% hydrogen bromide solution were charged to a reactor. 170 grams of 30% hydrogen peroxide was added drop-wise at 0° C. over 2 hours. The reaction temperature was controlled at 0-30° C. After reaction, the product was precipitated as a solid, and then the reaction mixture was quenched with 10% sodium sulfite. After filtration and drying, 142 g of high purity (95%, LC Area) of 3,4,5-tribromo-1H-pyrazole was obtained.

### Example 2. Bromine/Sodium Hydroxide as a Halogenation Reagent

34 grams of pyrazole was dissolved in water and then sodium hydroxide was added at 0° C. to obtain the corresponding pyrazole sodium salt. Next, 239.7 g of bromine was added drop-wise at 0° C. over 2 hours. The reaction temperature was controlled at 20-40° C. After reaction, the product was precipitated as a solid, and then the reaction mixture was quenched with 10% sodium sulfite. After filtration and drying, 147 g of high purity (98%, LC Area) of 3,4,5-tribromo-1H-pyrazole was obtained.

### Example 3. Potassium Iodide/Sodium Sulfite as a Dehalogenation Reagent

100 grams of 3,4,5-tribromo-1H-pyrazole, 1.1 g of KI, and 62 g of Na₂SO₃ in 300 mL DMAc were reacted at 130-150° C. for 14 hours to completed reaction. After completion of the reaction, the reaction mixture was filtered, and then DMAc was distilled off under vacuum. Next, water was added to the crude product. The reaction mixture was stirred for 10 min. The product, 3,5-dibromo-1H-pyrazole, was precipitated as a solid. After filtration and drying, 68 g of high purity (98%, LC Area) of 3,5-dibromo-1H-pyrazole was obtained.

### Example 4. Coupling Reaction

22.6 grams of 3,5-dibromo-1H-pyrazole and 10.6 g of carbonate were dissolved in 33.9 g sulfolane at 30° C. Then, 44.4 g of 2,3-dichloropyridine were added and the mixture was reacted at 170-180° C. After reaction, the reaction mass was cooled to 80-85° C. Excess 2,3-dichloropyridine was removed by steam distillation (100-105° C.). After excess 2,3-dichloropyridine was removed, the reaction mass was further cooled to 25-30° C. and water was added and twice extracted with methyl tert-butyl ether (MTBE). The MTBE layers were combined and removed in vacuum to yield 33 g of (95%, LC Area) 3-chloro-2-(3,5-dibromo-1H-pyrazol-1-yl)pyridine, which could be used in subsequent steps.

### Example 5. Reaction in the Presence of a Grignard Reagent

33.7 grams of 3-chloro-2-(3,5-dibromo-1H-pyrazol-1-yl)pyridine was dissolved in Me-THF then iPrMgCl was added at 0° C. to yield the corresponding 3-chloro-2-(3,5-dibromo-1H-pyrazol-1-yl)pyridine magnesium salt. After 0.5 hours, dry CO₂ gas was bubbled through the reaction mixture. The reaction temperature was controlled at 20-40° C. After reaction, the reaction mixture was quenched with water, acidified to pH=1 with 1M HCl, and extracted with Me-THF three times. The combined organic phase was concentrated in vacuum. 32 g of high purity (90%, LC Area) 5-bromo-2-(3-chloro-pyridin-2-yl)-2H-pyrazole-3-carboxylic acid was obtained.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula V, wherein 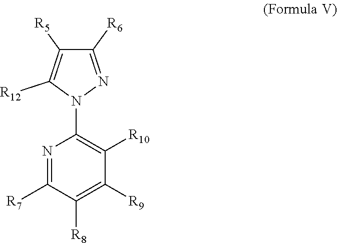
each of R₅-R₁₀ is independently selected from hydrogen and halogen; and
R₁₂ is an organic acid, the method comprising
I) forming a mixture comprising
A) a compound of Formula III, wherein 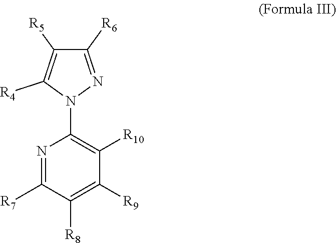
R₄ is a halogen; and
each of R₅-R₁₀ is independently selected from hydrogen and halogen;
B) a solvent;
C) a carbonyl-containing compound;
D) a compound comprising a metal; and
E) optionally an additive; and
II) reacting the mixture.

2. The method of claim 1, wherein the compound comprising a metal is selected from a Grignard reagent and a lithium-containing compound.

3. The method of claim 2, wherein the Grignard reagent is selected from MeMgCl, iPrMgCl, iPrMgBr, EtMgCl, and combinations thereof.

4. The method of claim 3, wherein the Grignard reagent is iPrMgCl.

5. The method of claim 2, wherein the lithium-containing compound is nBuLi.

6. The method of claim 1, wherein the solvent is selected from THf, toluene, 1,4-dioxane, Me-THf, and combinations thereof.

7. The method of claim 6, wherein the solvent is THF.

8. The method of claim 1, wherein the carbonyl-containing compound is selected from dimethyl carbonate, N,N-dimethyacetamide, carbon dioxide, and combinations thereof.

9. The method of claim 8, wherein the carbonyl-containing compound is carbon dioxide.

10. The method of claim 1, wherein the method step II) of reacting the mixture occurs at a reaction temperature in the range of about 0° C. to about 60° C.

11. The method of claim 10, wherein the method step II) of reacting the mixture occurs at a reaction temperature in the range of about 0° C. to about 30° C.

12. The method of claim 1, wherein R₅, and R₆ of Formula III are each independently hydrogen.

13. The method of claim 1, wherein the compound of Formula III is prepared according to a method comprising
I) forming a mixture comprising
A) a compound of Formula II, wherein 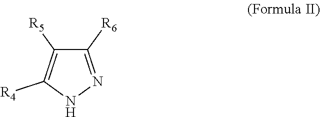
each of R₄, R₅, and R₆ is independently selected from hydrogen and halogen;
wherein at least one of R₄, R₅, and R₆ is hydrogen; and
wherein the compound of Formula II is prepared according to a method comprising
i) forming a mixture comprising
a) a compound of Formula I, wherein 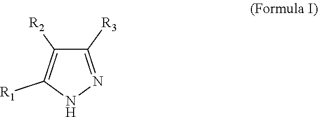
each of R₁, R₂, and R₃ is independently a halogen;
b) optionally a dehalogenation reagent;
c) a reducing agent; and
d) a solvent; and
ii) reacting the mixture;
B) a compound of Formula IV, wherein 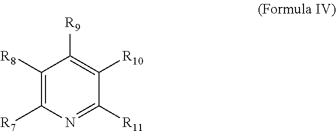
each of R₇-R₁₁ is independently selected from hydrogen and halogen;
C) a solvent;
D) an inorganic base; and
E) optionally an additive; and
II) reacting the mixture.

14. The method of claim 13, wherein the inorganic base is selected from powder sodium hydroxide, powder potassium hydroxide, potassium carbonate, potassium phosphate, powder sodium methoxide, powder potassium t-butoxide, and combinations thereof.

15. The method of claim 13, wherein the solvent C) is selected from heavy aromatic solvents, heavy aromatic solvent S150, heavy aromatic solvent S200, acetonitrile (MeCN), toluene, N,N-dimethylformamide (DMF), N,N-dimethylacetamide (DMAc), N-methyl-2-pyrrolidone (NMP), diglyme, triglyme, sulfolane and combinations thereof

16. The method of claim 13, wherein the additive is selected from potassium iodide, a phase transfer catalyst, and combinations thereof.

17. The method of claim 16, wherein the phase transfer catalyst is selected from butyl ammonium chloride, tetra butyl ammonium bromide, aliquat-336, 18-crown-6, and combinations thereof.

18. A method of preparing a compound of Formula II, wherein 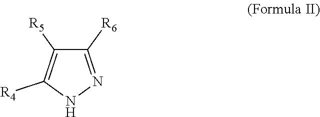
each of R₄, R₅, and R₆ is independently selected from hydrogen and halogen; and
wherein at least one of R₄, R₅, and R₆ is hydrogen, the method comprising
I) forming a mixture comprising
A) a compound of Formula I, wherein 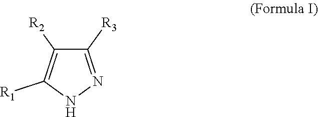
each of R₁, R₂, and R₃ is independently a halogen; and
wherein the compound of Formula I is prepared according to a method comprising
i) forming a mixture comprising
a) pyrazole or a pyrazole derivative;
b) a halogenation reagent;
c) a reaction solvent comprising water and an organic solvent; and
d) optionally an inorganic base; and
ii) reacting the mixture;
B) optionally a dehalogenation reagent;
C) a reducing agent; and
D) a solvent; and
II) reacting the mixture.

19. The method of claim 18, wherein the solvent D) is selected from acetic acid, water, toluene, N,N-dimethylformamide, N,N-dimethylacetamide, and combinations thereof.

20. The method of claim 18, wherein the reducing agent is selected from sodium sulfite, sodium bisulfite, sodium hyposulfite, sodium thiosulfate, sodium hydrosulfide, sodium sulfate, and combinations thereof.
